(19) United States
(12) Patent Application Publication
(10) Pub. No.: US 2012/0328672 A1
(43) Pub. Date: Dec. 27, 2012

(54) COMPOSITION AND METHOD OF SEPARATING BENTONITE INTO PARTICLES HAVING DISCRETE SIZE AND DENSITY RANGES CAPABLE OF BINDING BIOLOGICAL TOXINS AND CHEMOTHERAPEUTIC AGENTS.

(76) Inventors: Craig Conrad, Parker, TX (US); Donald Jones, Sylacauga, AL (US)

(21) Appl. No.: 13/525,267

(22) Filed: Jun. 15, 2012

Related U.S. Application Data
(63) Continuation of application No. 61/497,071, filed on Jun. 15, 2011.

(51) Int. Cl. A61K 33/06; A61P 1/12; C01B 33/26; A61K 9/14

(52) U.S. Cl. 424/400; 424/684; 423/328.1

(57) ABSTRACT

A low heavy metal (i.e. cadmium, mercury) containing calcium aluminum silicate product produced by the method of Air Classification. The method comprises using an air classification system for separating a cadmium containing calcium aluminum silicate feed stock into at least a first fraction and a second fraction. The first separation fraction contains material having an average particle size over 100 um, and the second fraction contains material having an average particle size under 100 um.

### RELATED APPLICATIONS

This application claims priority to U.S. Provisional Patent Application No. 61/497,071, filed on Jun. 6, 2011 having Craig Conrad and Don Jones listed as inventors.

### FEDERALLY SPONSORED RESEARCH

Not Applicable.

### JOINT RESEARCH AGREEMENTS

Not Applicable.

### SEQUENCE LISTING

Not Applicable.

### BACKGROUND OF THE INVENTION

The present invention relates to an industrial mineral resource isolation system and the resultant isolated materials. In particular, the invention relates to an industrial material separation system capable of isolating industrial quantities of material having distinct physical and chemically properties. The industrial isolation system utilizes a modified air stream cyclone apparatus to isolate useful material based upon particle size ranges and densities of the starting material. The method is useful for isolating compositions having altered physical and chemical properties when compared to the original starting mineral. In preferred embodiments, over 95% of a specific range of material can be isolated on an industrial scale. The size range and density of the isolated mineral can be modified depending on users needs and the types of materials isolated. Additionally, this system can be used to remove chemical contaminants from material. In one embodiment, the system was used to isolate a range of particle sized material having lower cadmium concentrations when compared to the bulk feed material. In another embodiment, the industrial method has been shown to isolate size ranged particles that increase the bulk density while simultaneously decreasing the time of wetting with water of a substantially anhydrous calcium aluminosilicate (“CAS”) mineral powder. One method comprises dry milling the powder in a media mill prior to air classification. The process enables improved electrostatic handling characteristics for specific sized particles of the treated CAS minerals with respect to bulk material handling systems. In another preferred embodiment, the industrial method allowed the isolation of a size density product from bentonite clay that adsorbed a biological toxin more efficiently than isolation products having different sized density ranges.

### Metal Contamination of Clay

Clay is a general term used to describe combinations different types of naturally formed minerals in the Earth's crust. Clay is a widely distributed, abundant mineral resource of key industrial importance for a vast variety of uses (e.g. from building materials to medicine). In both value and volume of annual production, clays are one of the leading minerals worldwide. There are over 4,000 different types of identified mineral clays, each of these different types of clay minerals have unique chemical structures and functional chemistry. Some metals such as Cadmium and Mercury are toxic to mammals. The product produced by the industrial method of this invention contains lower Cadmium and Mercury concentrations when compared to the stock feed composition.

Clay minerals can be formed or originate beneath the surface with a hypogene process. These hypogene formation processes can result from the action of gases, vapors, or solutions that originate below the surface and are forced upward through rocks in the Earth's crust. Although not wanting to be bound by theory, the invaded rocks contribute many of the metal elements found in clay minerals. Only a few metal elements found in clay are derived from deeper magma metal sources.

Formation of clay occurs at temperatures in the Earth that range from slightly below 100° C. to over 450° C. The main materials removed from the crustal rocks and used during the formation of the clay are alumina, silica, alkali or alkaline earth elements, and iron. The local environment may have been acid, neutral or alkaline depending upon the pH of the invaded rocks and that acidity of the vapors from the magma. Moreover, the minerals found in the in the wall rock of the conduits of fumaroles, geysers, and volcanic vents can be transferred to clay.

Additionally, processes involving compressed vapors or mixed liquids and vapors may alter rock-forming minerals of clay in cavities in pegmatic dikes or in igneous masses. Although not wanting to be bound by theory, the most extensive hypogene clay deposits may have resulted from the action of thermal waters, wherein some transfer of metals are limited to the borders of metal carrying veins and others are distributed over a wide area.

Although not wanting to be bound by theory, the particle size of certain clay composition during formation may actually lead to a different structural chemistry of the composition. Because clays were formed millions of years ago, it is nearly impossible to know for sure the geological conditions the created today's clay deposits. However, some experimental processes may indicate that the size and shape of a clay particle may transfer structure/function capabilities. For example, Chunyi et al., form the Lawrence Berkeley National Laboratory in Berkeley, Calif. published an article titled: “Particle Size Dependent Chemistry from Laser Ablation of Brass,” in Anal. Chem., 2005, 77 (20), pp 6687-6691. This paper showed how the proportion of zinc and copper in particles formed by laser ablation of brass was found to vary with the particle diameter. Energy-dispersive X-ray analysis showed that smaller particles were zinc enhanced while larger particles were composed mostly of copper. A model based on condensation of vapor onto large droplets ejected from a melted liquid layer may describe a change in particle composition versus size. The Particle size effect has also been describe as the thermodynamic solubility constant is defined for large monocrystals. More specifically, it may be that the solubility will increase with decreasing size of solute particle (or droplet) because of the additional surface energy. This group postulated that the effect of the particle size on solubility constant could be quantified. Theoretically, a solubility constant for the solute particles with the molar surface area having a molar surface area tending toward zero (i.e., when the particles are large), γ is the surface tension of the solute particle in the solvent, Aₘ is the molar surface area of the solute (in m²/mol), R is the universal gas constant, and T is the absolute temperature.

Clay mineral deposits are excavated and processed from clay mines found around the globe. As expected, each clay mines has a unique composition of impurities based on the local geology, and/or prior industrial use of the land. For example, clay mines located in areas that contain high levels of metals in the soil may be useful as building materials but not suitable for human consumption. Although not wanting to be bound by theory, it may be possible to isolate specific ranges of particle sizes of clay having different chemistries and different concentrations of various metals. A product and process for isolating clay particles having more or less metal concentrations could open different markets for different clay particle having defined chemistry, based upon the size/density range of particles within the isolated group.

Various clay samples from a roughly 14,000 square acre montmorillonite clay mine source in Jackson, Miss. was utilized as examples for products and processes of this invention. More specifically, a BASF owned Clay mine in Jackson, Miss. has unusually low levels of mammalian toxins and metals that are normally present in other clays that are found world wide.

Various adsorbents have been used, such as aluminas, zeolites, silicas, phyllosilicates, bentonite, activated charcoal, and montmorillonite. In particular, a hydrated sodium calcium aluminosilicate produced by BASF Corporation was used to “adsorb” and inactivate aflatoxin. See, U.S. Pat. No. 5,178,832 to Timothy D. Phillips, et al.; U.S. Pat. No. 5,165,946 to Dennis R. Taylor, et al.; and K. Pimpukdee, Feed & Livestock, pages 40-43, December 2003/January 2004; United States Patent Application 20080026079 submitted by Carpenter; Robert Hunt; et al. Jan. 31, 2008, titled “Calcium aluminosilicate pharmaceutical;” and United States Patent Application 20080008763 submitted by Phillips; Timothy D; et al. on Jan. 10, 2008, and titled “Composition and methods for the enterosorption and management of toxins;” the entire content of each of which is hereby incorporated by reference.

This invention utilized an industrial separation system to isolate a composition having a very different metal profile from a general bentonite product, (“the Feed Composition”). This industrial process allowed about 10 tons per hour of stock bentonite feed composition having a particle size range from about less than one-micrometer to over one-hundred-and-fifty-micrometers (<1 um to >150 um) to be classified into several product streams having particle sizes ranging from (<19 um), (20 um-60 um), (60 um to 100 um) and (>100 um). Each of the size ranges containing over 95% of the size ranged material. Moreover, different particle sized fractions were tested and found to contain a unique metal composition profile when compared to the feed material. Moreover, the isolated size and density ranges show how to isolate a specific size range of particles having dramatically different chemical and mechanical features. One of ordinary skill in the art would find it to be unexpected to have an isolation of different ranges of particle sizes to have different chemistries. Moreover, one having ordinary skill in the art will understand the metal content and composition of a structure can dramatically affect its chemical function. The a user of this technology can concentrate large quantities of reactive particles and decrease production costs. Moreover, custom particle sized products can be isolated and functions enhanced using this industrial isolation technology.

### Sifter Technology

A sieve, or sifter, separates wanted elements from unwanted material using a woven screen such as a mesh or net. The word “sift” derives from sieve. Sieving is a simple and convenient technique of separating particles of different sizes. However, there are mechanical limits to mechanical sifting. More specifically, screens smaller than about 30 um do not exist. A small sieve such as that used for sifting flour has very small holes that allow only very fine flour particles to pass through. The coarse particles are retained in the sieve or are broken up by grinding against the screen windows. Depending upon the types of particles to be separated, sieves with different types of holes are used. Separating tea leaves from tea is not considered to be sieving.

Mechanical vibratory sieves also commonly referred to as gyratory separators or screening machines, are a traditional part of processing dry bulk powders. They classify materials by separating them by particle size through a screen mesh. Using a combination of horizontal and vertical movements by means of a vibratory motor, they spread the material over a screen in controlled flow patterns and stratify the product. There are three main functions a vibratory sieve or separator can achieve: (a) Check/safety screening—used for quality assurance by checking for foreign contaminants and oversized material and removing them from the product; (b) grading/sizing screening—used to grade or classify material into different particle sizes; (c) Recovery screening—used to recover valuable materials in the waste stream for re-use.

Although not wanting to be bound by theory, most machines vibrate at 1400 rpm, but by separating the motor from the rubber suspension in this type of design, it became possible to increase the operating speed of the machines up to 2800 rpm with high out-of-balance forces. This development led to increased efficiency of the sieve, enabling smaller diameter machines to be used without adversely affecting performance. For example, a 22″ diameter machine operating at 2800 rpm can significantly out-perform a 48″ diameter machine operating at 1400 rpm on materials that are traditionally difficult to screen.

There are many different types of sifting systems. The Kason sifting system is a versatile, high-capacity centrifugal sifter used for screening powders and granular materials for the food and pharmaceutical industries. Material is gravity fed into the feed inlet and redirected by a feed screw into the cylindrical sifting chamber. Rotating, helical paddles in the chamber propel the material against the screen, while the resultant centrifugal force accelerates the particles through the apertures. The paddles, which never contact the screen, also break up soft agglomerates. Oversized particles and foreign material are ejected through the oversized discharge spout.

Additionally, the Sweco sifting system is a round screening device that vibrates around its center of mass due to eccentric weights on the upper and lower ends of the motion-generator shaft. Rotation of the top weight creates vibration in the horizontal plane, causing material to move across the screen cloth to the edges. The lower weight tilts the machine, causing vibration in the vertical and tangential planes. The spiral screening pattern can be variably controlled by the angle of lead given to the lower weight in relation to the upper weight. The speed and spiral pattern can be set by the operator for maximum throughput and screening efficiency of any screenable product.

Although not wanting to be bound by theory, there are at least two related problems with screening technology. The first is a build up of static electricity, and the second is isolation of a specific particle size. Depending on the material being sifted, a build up of static electricity can cause smaller particles to be electrostatically attached to larger particles. As shown in FIG. 11, even though the larger particle was separated using a screen, the electrostatic attraction allowed many smaller particles to remain, which may alter the physical and chemical properties of the desired product.

### Air Cyclones Classifier

In general, the air stream cyclone apparatus has some obvious advantages over screen shaker technology. One advantage being high efficiency and another being high throughput. The Air Classifier is easily maintained because there are relatively few moving parts. The Air Classifier is easy to clean and builds up minimal static electric charge when compared to screen shaker units, as shown in FIG. 12.

The operation theory of the mineral resource material classification system is based on a vortex motion where the centrifugal forces act on each particle and therefore causes the particle to move away from the cyclone axis towards the inner cyclone wall. However, the movement in the radial direction is the result of two opposing forces where the centrifugal force acts to move the particle to the wall, while the drag force of the air acts to carry the particles into the axis. As the centrifugal force is predominant, a separation of different particle sizes takes place.

Powder and air pass tangentially into the cyclone at equal velocities. Powder and air swirl in a spiral form down to the base of the cyclone separating the powder out to the cyclone wall. Powder leaves the bottom of the cyclone via a locking device. The clean air spirals upwards along the centre axis of the cyclone and passes out at the top.

The centrifugal force each particle is exposed to can be seen in this equation:

C=m×Vt2/r(16)

Where:
C=centrifugal force
m=mass of particle
Vt=tangential air velocity
r=radial distance to the wall from any given point

From this equation it can be concluded that the higher particle mass, the better efficiency. The shorter way the particle has to travel the better efficiency, and the closer the particle is to the wall the better efficiency, because the velocity is highest and the radial distance is short.

However, time is required for the particles to travel to the cyclone wall, so a sufficient air residence time should be taken into consideration when designing a cyclone. From above equation it is evident that small cyclones (diameter less than 1 m) will have the highest efficiency, a fact generally accepted.

However, the big tonnage dryers in operation in the dairy industry nowadays would require many cyclones (a cyclone battery). As each cyclone has to have an outlet for powder in form of a rotary valve, pneumatic valve or flap valve, this means that there is a big risk of air leaks which will reduce the cyclone efficiency. The small cyclones can also be connected to one central hopper, and only one valve is then necessary. This means however, that unless there is exactly the same pressure drop over each cyclone, air and powder will pass from one cyclone to another via the bottom outlet. This will result in decreased efficiency and increased powder loss. Cleaning the many small cyclones is a problem, as it is a time consuming job, and with the many corners there is a risk of a bacterial infection.

For above reasons the cyclones have become bigger and bigger and are now constructed with diameters of 2.5-3 m, each handling 25,000-30,000 kg of air/h.

When designing a cyclone various key figures should be taken into account in order to obtain the highest efficiency. This is achieved if:
cyclone diameter/exit duct diameter≈3
cyclone height/exit duct diameter≈10
Air through-put (velocity Vₜ) and increased pressure drops will also increase the efficiency, but the energy requirement will increase simultaneously, so in general the upper limit is about 175-200 mm WG for skim milk powder. 140-160 mm WG is the maxi-mum for whole milk in order to avoid deposits and final blocking

In most cases rotary valves are used as air lock and product discharge at the bottom of the cyclone. The conical type allowing for easy adaption of the gap between the housing and the rotor could be preferred to reduce powder loss.

In order to know a cyclone's efficiency the following terms have to be defined:
a) The critical particle diameter;
b) The cut size; and
c) The overall cyclone efficiency.

The critical particle diameter is defined as the particle size that will be completely removed from the air flow (100% collection efficiency). Although not wanting to be bound by theory, there is no sharply defined point where a particle size is 100% separated or 100% lost, wherein the critical particle diameter is not an extremely beneficial value.

The cut size is defined as the size for which 50% collection is obtained and is a much better value for stating the efficiency of cyclones. To determine a cyclone's cut size, grade efficiency curves are worked out by systematically operating a cyclone with a uniform particle size dust.

The overall cyclone efficiency is the number obtained when handling a product of definite size distribution. Knowing the grade efficiency curve of the cyclone and the product size distribution of the powder passing to the cyclones, the overall efficiency can be calculated (i.e. the powder loss can be predicted). Another method of learning the cyclone efficiency is by a simple powder loss measurement after the cyclone.

A very small fraction of the out-going air is passed through a high-efficient mini cyclone or through micro dust filters. The amount of powder collected is directly proportional to the powder loss, which will mainly be a result of:
Feed with low solids or feed containing air
High outlet air temperature
Low particle density (as a result of the above, for example)
Leaking product outlet from old non-adjusted rotary valves
Blocked cyclone
Change in drying parameter resulting in decrease of mean particle size
Old cyclones, dented due to heavy beating to avoid blockings

U.S. Pat. No. 4,257,880 titled “Centrifugal Air Classifying Apparatus,” issued to Jones on Mar. 24, 1981 (the “Jones '880 Patent) was a leap forward in cyclone air classifying design. More specifically, the Jones '880 Patent comprised a centrifugal air classifying apparatus for use with a cyclone type centrifugal separator and a fan for drawing air from the separator and returning it at superatmospheric pressure to the classifying apparatus, wherein the classifying apparatus has a rotary particle rejector in its upper portion for classifying material fed into a rising and rotating column of air outwardly surrounding the rejector. A first primary annular sealing zone is provided adjacent the tops of the rejector blades and a secondary annular sealing ring is provided at an intermediate location between the upper and lower ends of the blades. The entire Jones '880 patent is incorporated herein by reference.

### SUMMARY

One aspect of the current invention is a low cadmium containing calcium aluminum silicate product produced by the method of Air Classification. The Air classification method comprises the steps of using an air classification system for separating a cadmium containing calcium aluminum silicate feed stock into at least a first fraction and a second fraction, wherein the first fraction contains material having an average particle size over 100 um, and the second fraction contains material having an average particle size under 100 um. In one preferred embodiment, the rotor speed of the Air Classifier is set at about 1125 and the fan speed is set at about 4,400. Additionally, the feed rate is set at about 18%. In another preferred embodiment a second air classification separation of the second fraction containing material having an average particle size under 100 um is completed. The resultant third fraction and a fourth fraction, wherein the particles having an average particle sized greater than 20 um are found in the third fraction, and an average particle sizes less than 20 um are found in the fourth fraction.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a side elevation view of a centrifugal type air classifying system for separating very fine particles from a mixed particle size feed;

FIG. 2 shows a top plan view of the system illustrated in FIG. 1;

FIG. 3 shows an elevation view of the system of FIG. 1, viewed from the right of FIG. 1;

FIG. 4 shows a cross section of the Air Classification system;

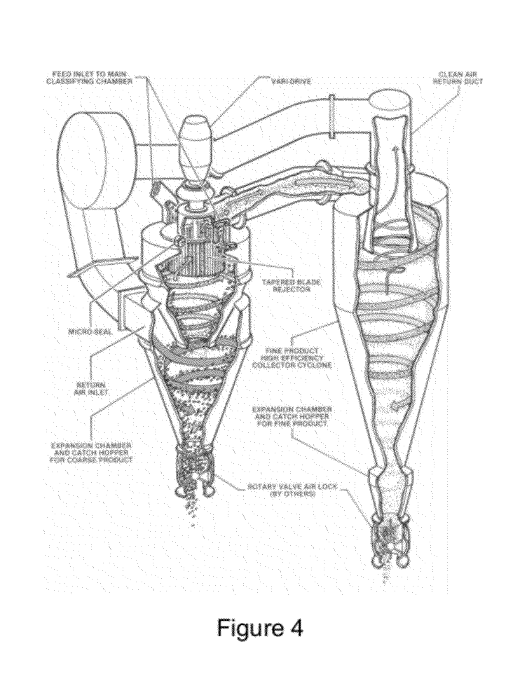

FIG. 5 shows a multi-pass system for classifying fine and coarse particles of Calcium Aluminum;

FIG. 6 shows the direct metal analysis of stock feed and air classified particles;

FIG. 7 shows the basic structure of clay;

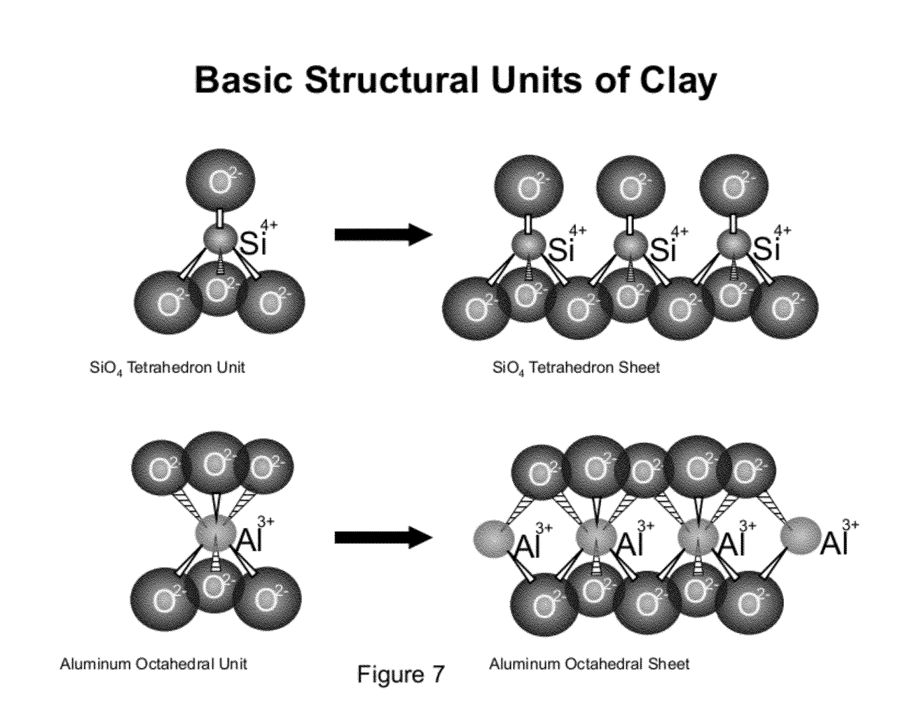

FIG. 8 shows the basic structure of Bentonite 2:1 Clay;

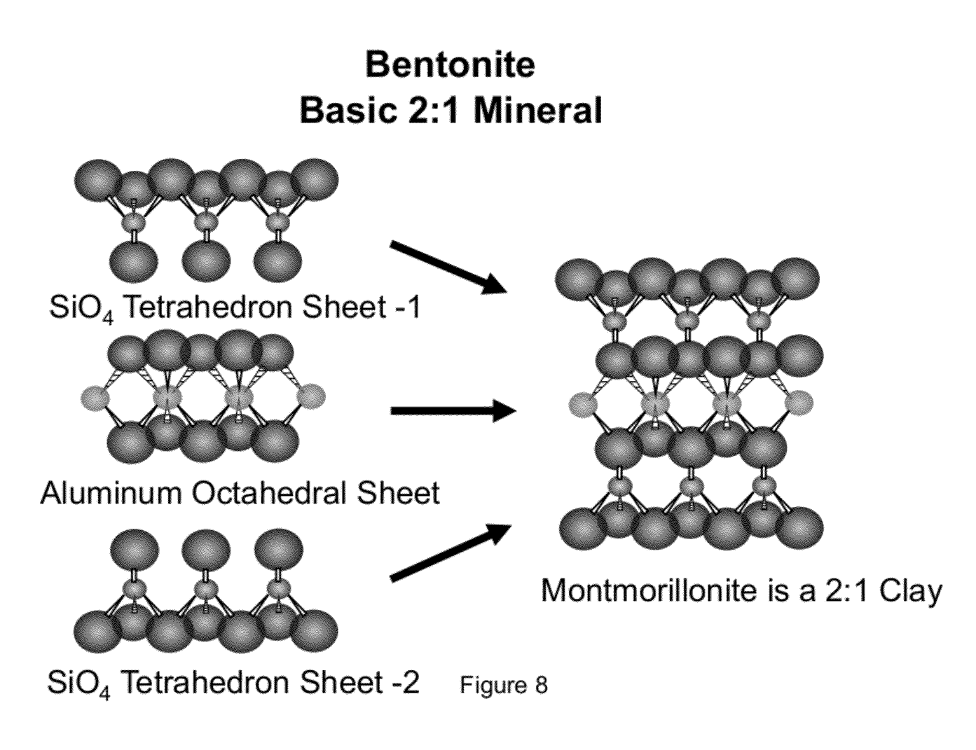

FIG. 9 shows the Stacked Platelet Structure of Bentonite Clay;

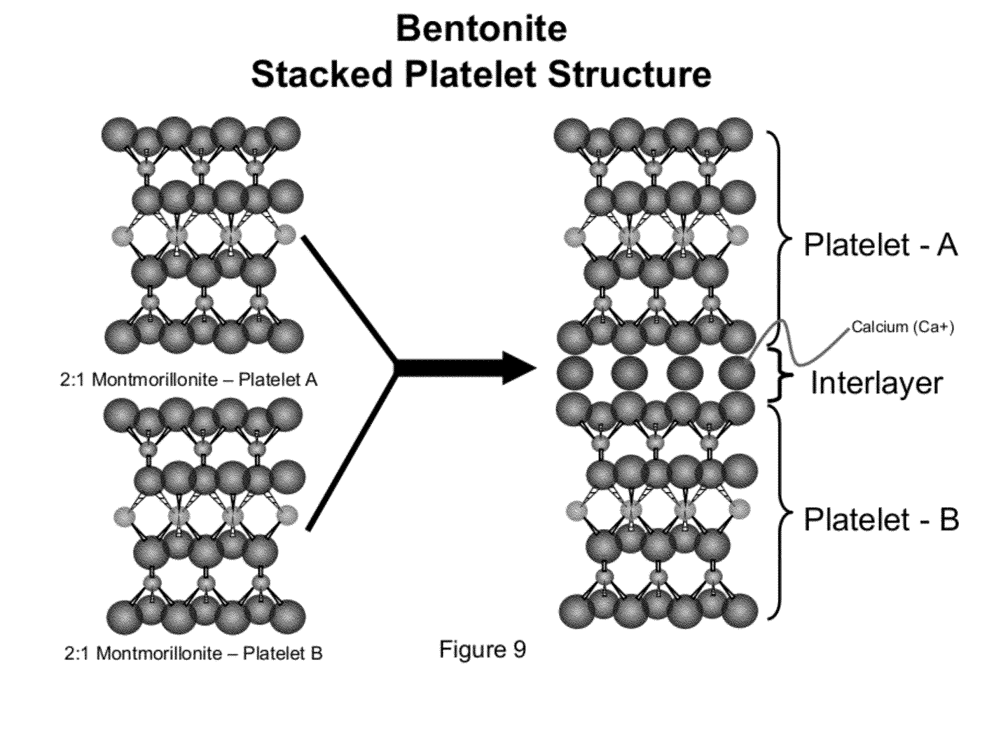

FIG. 10 shows the Stacked Platelet Structure of Bentonite Clay;

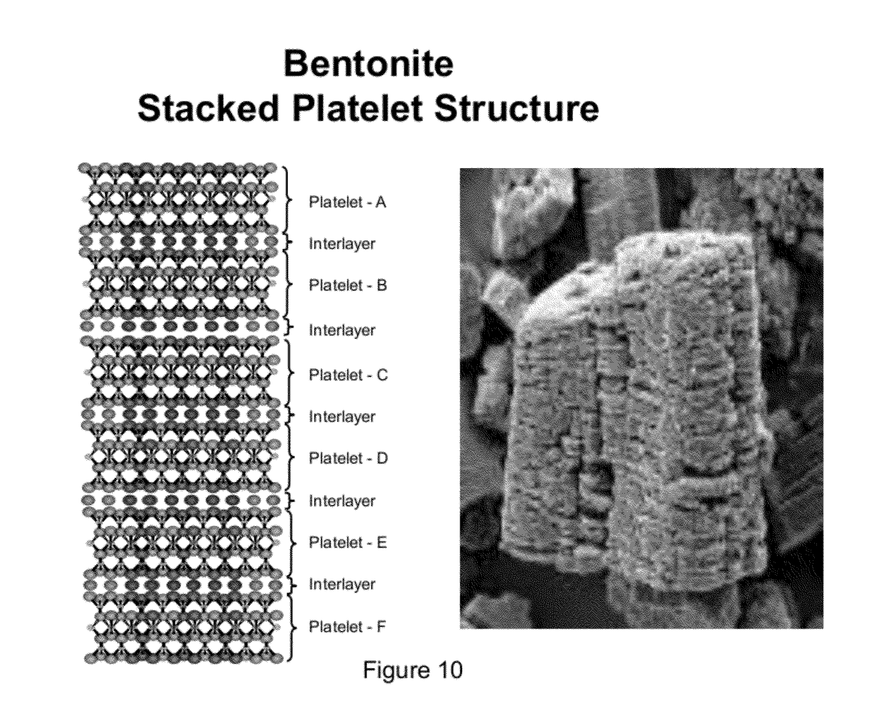

FIG. 11 shows sizing using a screen mesh;

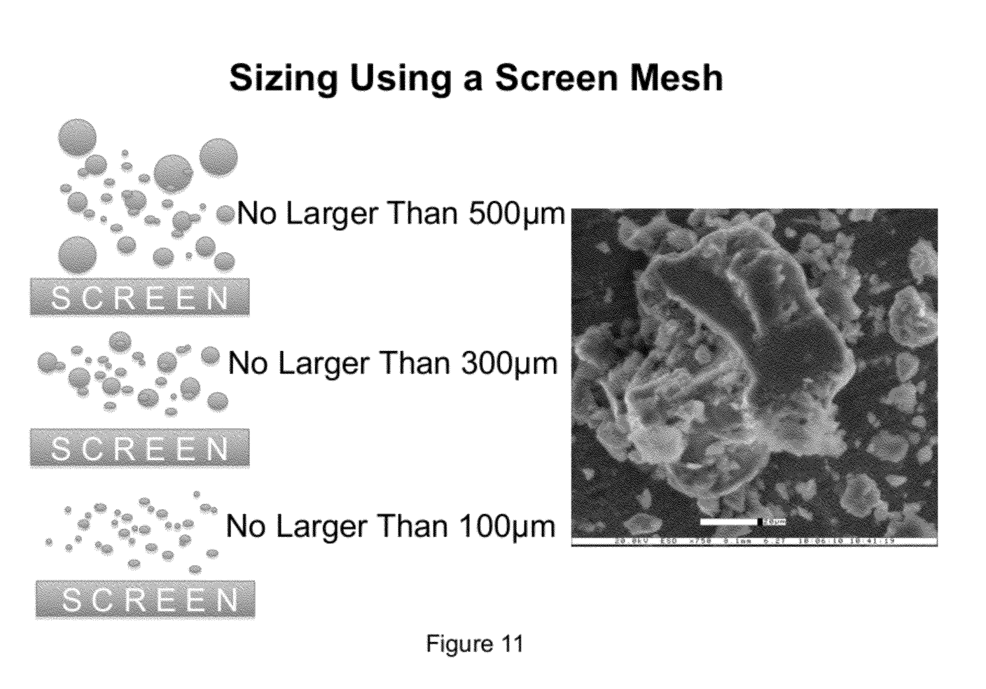

FIG. 12 shows sizing using an Air Classification System;

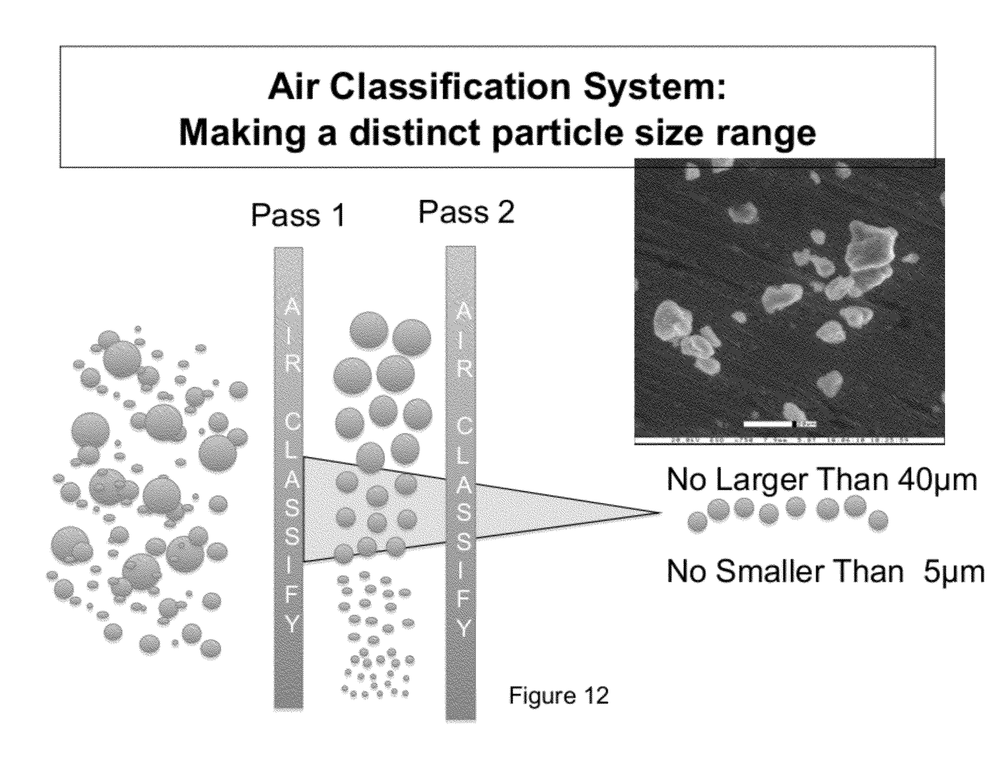

FIG. 13 shows surface area of small particles compared to larger particles.

FIG. 14 shows how static builds up with bentonite particles on a watch glass compared with little static when separated using the invention.

FIG. 15 shows a simulated Beckman Coulter LS Particle Size Analyzer data sheet of Bentonite Clay before and after processing, wherein over 95 percent of the processed material falls within a specific particle size range.

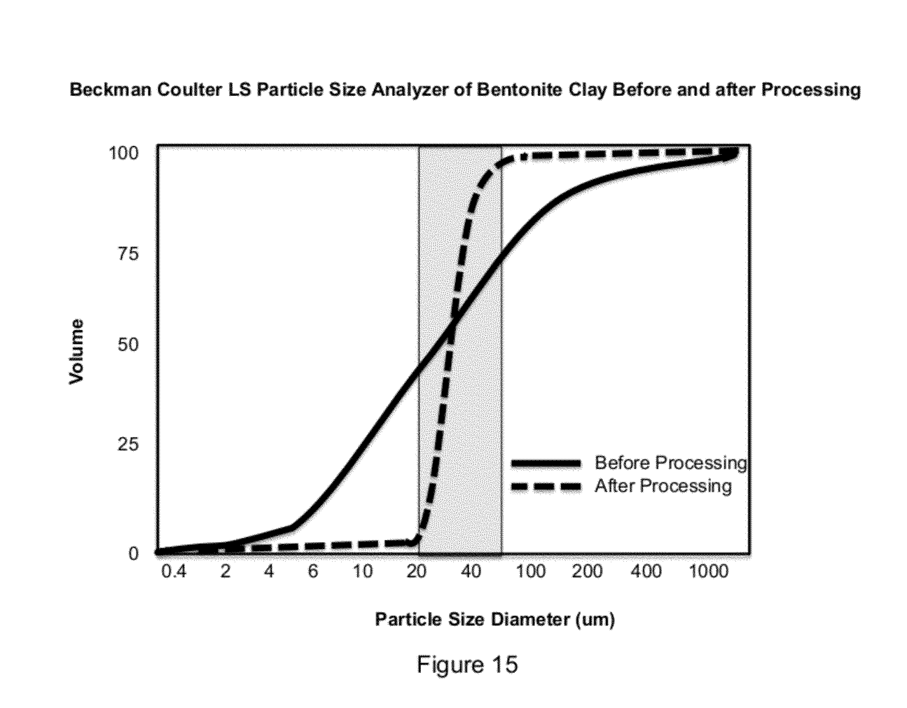

FIG. 16 shows AFB₁ adsorption isotherms on particles having district size ranges.

### DETAILED DESCRIPTION

Before describing the present invention in detail, it is to be understood that this invention is not limited to particular compositions or methods for making compositions, which may vary. It is also to be understood that the terminology used herein is for the purpose of describing particular embodiments only, and is not intended to be limiting. In addition, before describing detailed embodiments of the invention, it will be useful to set forth definitions that are used in describing the invention. The definitions set forth apply only to the terms as they are used in this patent and may not be applicable to the same terms as used elsewhere, for example in scientific literature or other patents or applications including other applications by these inventors or assigned to common owners. Additionally, when examples are given, they are intended to be exemplary only and not to be restrictive.

It must be noted that, as used in this specification and the appended claims, the singular forms “a,” “an” and “the” include plural referents unless the context clearly dictates otherwise. Thus, for example, reference to “a pharmacologically active agent” includes a mixture of two or more such compounds, reference to “a base” includes mixtures of two or more bases, and the like.

In describing and claiming the present invention, the following terminology will be used in accordance with the definitions set out below.

“Active agent,” “pharmacologically active agent,” “composition,” and “drug” are used interchangeably herein to refer to compositions and drugs that are useful as a preservative and additive for food and feed. The terms also encompass pharmaceutically acceptable, pharmacologically active derivatives and analogs of such drugs, including, but not limited to, salts, esters, amides, prodrugs, active metabolites, inclusion complexes, analogs, and the like. Therefore, when the terms “active agent,” “pharmacologically active agent”, or “drug” are used, it is to be understood that applicants intend to include the active composition per se as well as pharmaceutically acceptable, pharmacologically active salts, esters, amides, pro-drugs, active metabolites, inclusion complexes, analogs, etc., which are collectively referred to herein as “pharmaceutically acceptable derivatives.”

The present invention pertains to compositions and methods of preventing microbial by adding a preservative and additive for to a hydrogel. Some of the toxins that occur in the environment are the aflatoxins, which are a group of carcinogenic mycotoxins produced primarily by Aspergillus flavus and Aspergillus parasiticus fungi and are often detected in foods and agricultural commodities. These compounds are heat stable and can survive a variety of food processing procedure; thus aflatoxins can occur as “unavoidable” contaminants of most foods and livestock feeds. Of four naturally occurring aflatoxins (B₁, B₂, G₁, and G₂), aflatoxin B₁ is the most toxic. In addition, several studies suggest that low-level exposure to aflatoxins may cause suppression of the immune system and increased susceptibility to disease.

One aspect of the present invention pertains to various low cadmium containing clays capable of relieving symptoms of diarrhea by killing, or inhibiting the growth of, harmful microorganisms and simultaneously inactivates mycotoxins, such as aflatoxins, present as contaminants in the gut that may lead to symptoms of diarrhea. The clay is an adsorbant having structure-selective affinities to various mycotoxins, such as aflatoxins, thus inactivating the mycotoxins present in human foods and animal feeds. Although not wanting to be bound by theory, the adsorbed or absorbed acid is believed to be available from the acidified clay to kill harmful microorganisms present as contaminants in human foods and animal feeds.

The term “complex,” as used herein, denotes a composition wherein individual constituents are associated. “Associated” means constituents are bound to one another either covalently or non-covalently, the latter as a result of hydrogen bonding or other inter-molecular forces. The constituents may be present in ionic, non-ionic, hydrated or other forms.

The term “Manganese” as used herein, denotes an essential trace nutrient in all forms of life. The classes of enzymes that have manganese cofactors are very broad and include oxidoreductases, transferases, hydrolases, lyases, isomerases, ligases, lectins, and integrins. The reverse transcriptases of many retroviruses (though not lentiviruses such as HIV) contain manganese. The best-known manganese-containing polypeptides may be arginase, the diphtheria toxin, and Mn-containing superoxide dismutase (Mn-SOD). Mn-SOD is the type of SOD present in eukaryotic mitochondria, and also in most bacteria (this fact is in keeping with the bacterial-origin theory of mitochondria). The Mn-SOD enzyme is probably one of the most ancient, for nearly all organisms living in the presence of oxygen use it to deal with the toxic effects of superoxide, formed from the 1-electron reduction of dioxygen. Exceptions include a few kinds of bacteria such as Lactobacillus plantarum and related lactobacilli, which use a different non-enzymatic mechanism, involving manganese (Mn²⁻) ions complexed with polyphosphate directly for this task, indicating how this function possibly evolved in aerobic life. The human body contains about 10 mg of manganese, which is stored mainly in the liver and kidneys. In the human brain the manganese is bound to manganese metalloproteins most notably glutamine synthetase in astrocytes. Manganese is also important in photosynthetic oxygen evolution in chloroplasts in plants. The oxygen evolving complex (OEC) is a part of Photosystem II contained in the thylakoid membranes of chloroplasts; it is responsible for the terminal photooxidation of water during the light reactions of photosynthesis and has a metalloenzyme core containing four atoms of manganese. For this reason, most broad-spectrum plant fertilizers contain manganese.

### EXAMPLES

The following examples are provided to further illustrate this invention and the manner in which it may be carried out. It will be understood, however, that the specific details given in the examples have been chosen for purposes of illustration only and not be construed as limiting the invention.

### Example 1

One of the most dangerous operations involving standard particle sizing with mechanical sifting and mesh screens are the large amount of dust produced. Apart from the obvious and characteristic signs of silicosis observed in the lungs and damage to the hilar lymphatic glands, studies of several fatal cases have revealed damage to various sections of the pulmonary arterial tree. Clinical examination has reveled respiratory disorders (Emphysema and sometimes pleural damage), cardiovascular disorders and renal disorders as well as signs of adrenal deficiency.

In addition to the potential health hazards, mechanical sifting of products tends to allow materials being separated to build up a static charge, which decreases the final product's handleability. Moreover, mechanical sifters are expensive and contain many moving parts, which add considerable cost to manufacturing and the final cost of goods.

In an effort to minimize manufacturing costs and decrease the potential health risks of mechanically sifting bentonite or montmorillonite clay in an industrial setting, the inventors utilized Air Classification techniques to separate specific particle size ranges of bentonite clay then looked at the characteristics of the resultant products. The end-results were clay products having specific ranges of almost uniform particles sizes, with little or no dust occurring in the manufacturing plant. These uniform particle sizes proved to have better industrial handablility with little or no static charge buildup on the smaller particles. Moreover, the chemical analysis showed that the metal composition of different sized particles changed uniformly and reproducibly. This was an unexpected result because one of ordinary skill in the art would assume that particles having only a one-, two- or three-fold difference in particle size would have similar chemistry.

Using the air classification system described below to separate various particle sizes of calcium aluminum silicate, our results showed that the particle sizes in the ranges from below about 20 μm to about 100 μm have different chemical composition when compared to samples having a particle size form about 100 μm to 300 μm.

### Air Classifier

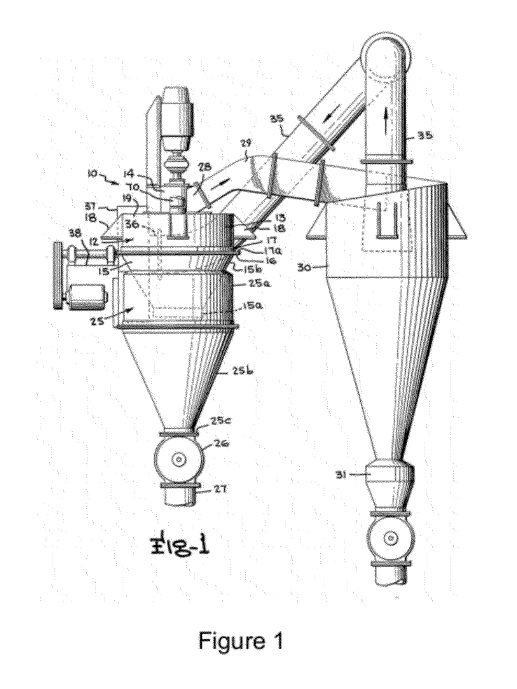

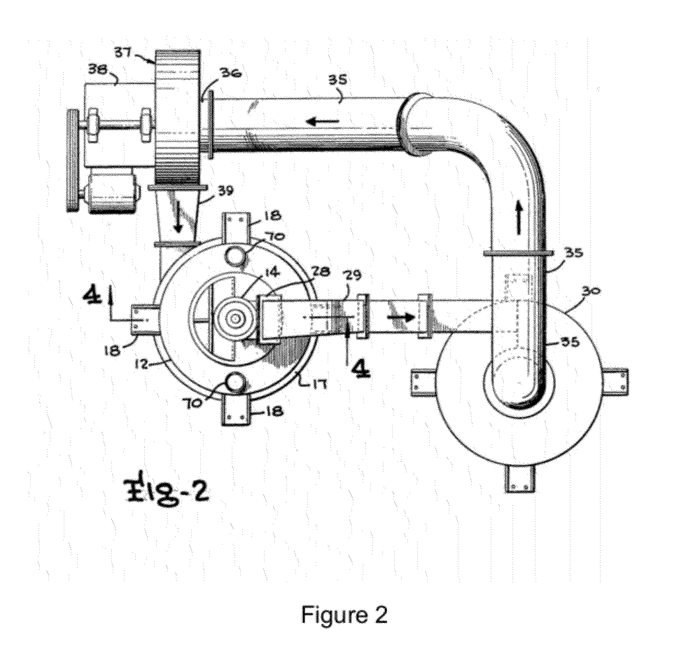

An air classifier system used in this example was built by Progressive Industries Inc, Sylacauga, Ala. was modified by adding ceramic tiles to the inside surface of the of the conical cyclone. More specifically, referring to the drawings, wherein like reference characters designate corresponding parts throughout the several figures, and referring particularly to FIGS. 1-3. This type of centrifugal type air classifying system is indicated generally by the reference character 10 and comprises an upper main classifying or rejector chamber and rotor assembly, which is indicated generally by the reference character 12, formed of a generally cylindrical rejector chamber portion 13 and an upwardly projecting cylindrical rotor support housing portion 14, assembled with a downwardly converging conical expansion chamber classifier, for example having a maximum practical diameter of about 44 inches, indicated generally at the reference character 15. The rejector chamber section 13 and classifier 15 are assembled, for example, by the horizontal flanges 16, 17a formed at the upper lip of the conical expansion chamber classifier 15 and the bottom of the annular angle iron mounting collar 17 at the bottom of the rejector chamber 13 and these assembled sections may be supported on suitable frame members by mounts such as the angle beam members 18. The rejector chamber 13 has a cylindrical outer or side wall 19 concentric with the vertical center axis of the rejector chamber and rotor assembly 12 and assembled to the mounting angle iron collar 17, and has a generally circular top wall 20 provided with a large center opening 21 on which is surmounted the support housing 14 of generally cylindrical configuration having an annular outwardly projecting intermediate mounting collar 22 fixed to the top support housing wall 20 by cap screws or similar fastenings indicated at 23.

The downwardly converging conical expansion chamber classifier 15 forms the lower unit of the main classifier 12 and provides an expansion chamber for the coarse particles which have been rejected from the upper rejector section 13 to be collected. The size of the opening, indicated at 15a, at the bottom of the cone portion 15b of the expansion chamber classifier section 15 is a variable which would be based on the bulk density of whatever material is to be fed to the classifier. The lower portion of the expansion chamber classifier cone 15 extends into the upper region of the cylindrical portion 25a of a receiver cone 25 having a lower cone shaped portion 25b, and the bottom opening 25c of this receiver cone 25 is connected to a conventional air lock 26 whose lower end connects to a coarse product discharge conduit 27 to lead the coarse product to the desired collection station.

The support housing 14 of the rotor assembly includes an upwardly inclined outlet formation 28 which connects by a duct 29 for example a 12″ diameter duct, to the upper portion of a fine particle classifier cyclone collector 30 disposed laterally from and alongside the assembly of the main classifying or rejector chamber and rotary assembly 12, expansion chamber cone classifier section 15 and cone receiver 25. The fines or light material, which have passed through the rotor assembly 14, later described in detail, are transported through the duct 29 to the fine particle classifier cyclone 30, which is specially designed to provide a screw top shaped so as to force the airstream carrying the light or fine particles to the cyclone collector 30 in a downwardly spiraling direction. The pressure drop and decreasing velocity at the upper portion of the cyclone collector 30 allows the fines or light particles to fall out as the air is pushed downward. The spinning air in the cyclone collector 30 causes the fines or light particles to be held to the outside portion of the cyclone collector, so that as the fines or light particles are pushed down to the point of discharge of the cyclone collector, they are dropped out as they enter the small cyclone or expansion chamber portion 31 of the cyclone collector 30 at the bottom. A vortex of cleaner air moves upwardly through the cyclone collector back to the return duct 35 at the upper center of the cyclone collector and returns this air from the cyclone collector to the inlet 36 of the main system fan 37 driven by a suitable fan motor 38, from whence the air is close-circuited back through the fan discharge duct 39 to the cone section 15 of the rejector and rotary assembly 12.

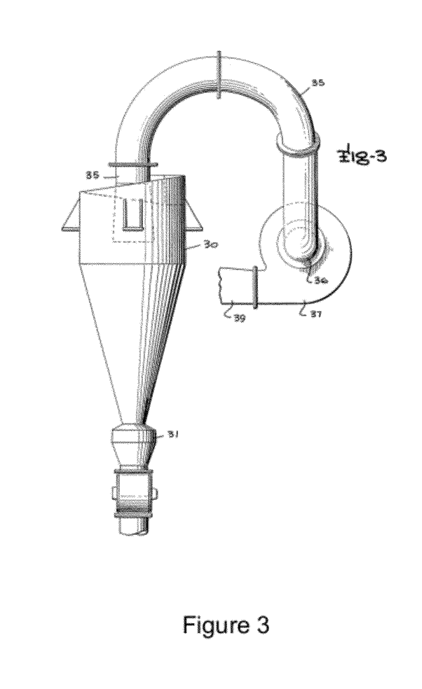

Referring now more particularly to FIG. 3 illustrating the details of the upper portion of the rejector chamber and rotor assembly 12 in larger scale, it will be seen that the support housing portion 14 is removably supported on the generally cylindrical housing 20 for the rejector chamber 13 by the annular collar or flange 22 lapping over the edges of the top wall portion 20a of the primary classifying chamber housing 20 and secured thereto by the cap screws 23 and that the support housing 14 in turn supports the generally vertically extending tubular cylindrical bearing housing 40. The support for the bearing housing 40 is provided by the upper annular collar or flange formation 41 lapping the top wall portion 14a of the rotor assembly support housing 14 bounding the opening 14b therein and fastened thereto by cap screws 42, and by a supporting spider formed of stabilizer tubes 43 and long cap screws 44 extending therethrough into tapped openings 45 in lower portions of the tubular bearing housing 40 and through the annular cylindrical lower wall portion 14c of the support housing 14 depending below the mounting flange or collar 22. The tubular bearing housing 40 has a pair of upper and lower bearing assemblies 46 journaling the vertical rejector shaft 47 concentrically therein, with a locking washer 48 and spanner nut 49 associated with each of the bearings 46. At the upper and lower ends of the tubular bearing housing 40 are a seal retainer cap, in the form of an annular plate, indicated at 50 secured to the annular end surfaces of the tubular bearing housing 40 by suitable cap screws and supporting an annular oil seal 51 bearing against the surface of the shaft 47.

Fixed to the lower end portion of the shaft 47 depending below the lower seal retainer cap 50 is a bottom spacer and hub member 52 which is fixed to the shaft 47 to be driven therewith by key 53 extending into aligned grooves or kerfs in the confronting portions of the shaft and the hub portion of the bottom spacer and hub member 52 and secured to the shaft by annular washer 54 and cap screw 55. The outer perimeter or edge of the spacer and hub member 52 has a bottom blade retainer 56 thereon, for securing the lower end portion of the vertically extending truncated wedge shaped rotor blades 57 in a generally cylindrical path outwardly of the depending annular cylindrical lower portion 14c of the support housing 14 and concentric with the axis of the shaft 47. The upper ends of the vertical rotor blades 57 are secured in position by an annular top blade retainer 58 and top spacer ring 59, which extends into and rotates within a downwardly opening annular cylindrical well 60 formed between the edge of the circular opening 21 in the top wall 14a of the housing 14 and the thickened root or inner portion of the annular mounting flange or collar 22 of the support housing 14.

The rejector chamber and rotor assembly includes a novel dual positive seal arrangement formed of a primary main positive seal indicated at 61 and a secondary safety seal arrangement indicated generally at 62. The primary main positive seal 61 is formed by the top blade retainer 58 projecting into the downwardly opening annular well 60 and by the positive seal ring 63 fixed to the thickened root or inner portion 22a of the support housing mounting collar 22 by cap screws 64 and lapping beneath the inner edge portion of the top blade retainer 58 and the top spacer ring 59 as shown, extending almost to the inner edges of the array of tapered blades 57. The secondary seal is formed by the annular secondary seal ring 65 fixed by cap screws 66 to the thickened lower end portion or rim 14d of the depending annular cylindrical lower portion 14c of the support housing 14 and extending to a location very close to the inner edges of the tapered rotor blades 57 with the outer edge of the secondary seal ring 65 lying in a circular path concentric with the axis of the rotor shaft 47 and of substantially the same diameter as the circular path of the outer edge of primary main positive seal ring 61.

The tapered blades 57 for the vertical blade rotary rejector are approximately ½″ wider at the top than at the bottom, causing the vertical blade rotary rejector to have varying tip speed with a fixed shaft speed or center line speed. This varying tip speed, being the highest at the top of the vertical blade rotary rejector, causes more air to flow at the top of the rejector chamber 13, providing for better dispersion and allowing the bottom portion of the vertical blade rotary rejector to recover a high percentage of the fine material entering the classifying device.

The material to be classified is delivered or supplied to the upper or primary classifying chamber 13 by a slide type air conveyor through, for example, a pair of diametrically opposite classifier feed tubes indicated generally at 70. This type of feed system causes the material to “float or swim” to the upper main or primary classifying chamber 13 so that the material is in a very fluffy or dispersed state prior to entering the rejector chamber 13. The rotating vertical tapered blade rotor assembly of tapered blades 57 causing greater air flow at the top of the main classifying chamber than at the bottom, causes the material to be classified to be held in suspension around the rotor by an upward column of air supplied from the closed system fan 37, for example, a 50 h.p. fan. The centrifugal spin of the upward column of air causes the coarser particles to be on the outside of the spin and the finer particles to be toward the center. Increasing the speed of the vertical blade rotary rejector permits increase of the resistance of the upcoming air or decrease in the velocity of the air moving across the rotary blade rejector, which causes the material taken through the rejector to be finer because the transport velocity is being decreased. When the rejector speed is decreased, the transport velocity is increased across the rejector, allowing it to take coarser or heavier products inwardly toward the center. The size of the products taken inwardly toward the center through the rotor rejector blades pass upwardly through the zone 71 outwardly surrounding the bearing housing 40 and inwardly of the depending annular cylindrical lower supporting housing portion 14c into the upper zone 72 and outwardly through the outlet fitting 28, to pass through the duct 29 to the spirally formed upper portion of the fine particle cyclone collector 30. In the cyclone collector 30, the pressure drop and decreasing velocity allows the fine or light particles to fall out as the air is pushed downward to the point of discharge where the fines drop out into the small cyclone or expansion chamber 31 at the bottom and thence through the outlet conduit connected to the bottom of the cyclone collector 30.

The assembly hereinabove described has a completely sealed to atmosphere having no leakage and requiring no dust collection equipment such as is required with other types of classifying devices heretofore marketed.

### Clay

The clay or mineral suitable for this invention include montmorillonite clay, phyllosilicate, Florisil®, bayerite, pseudoboehmite, alumina, silica gel, aluminum oxides, gibbisite, boehmite, and bauxite. The preferred clay used includes hydrated sodium calcium aluminosilicate (“HSCAS” clay commercially available as Novasil clay™ which is produced by BASF Corporation). A more preferred clay is F-100, which is also produced by BASF corporation.

For this example, a clay of hydrated sodium calcium aluminosilicate, (e.g. Montmorillonite clay™) having a distribution of particle size of in the range of about less than 1 microns to over 300 microns was utilized. The appearance of Montmorillonite clay™ is off white to tan colored and is a free flowing powder. The free moisture content is about 9%. The loose bulk density is 0.64 g/cc; the packed bulk density is about 0.80 g/cc; and the particle size distribution is about 5% of +100 mesh, 18% of +200 mesh, and 60% of −325 mesh. The clay is substantially free from dioxins (dioxin as used here refers to the toxic contaminant 2,3,7,8-tetrachlorodibenzodioxin (“TCDD”) which is used as an index of the presence of dioxins in food ingredient) in Montmorillonite clay above the detection limit of 0.33 parts per trillion (“ppt”).

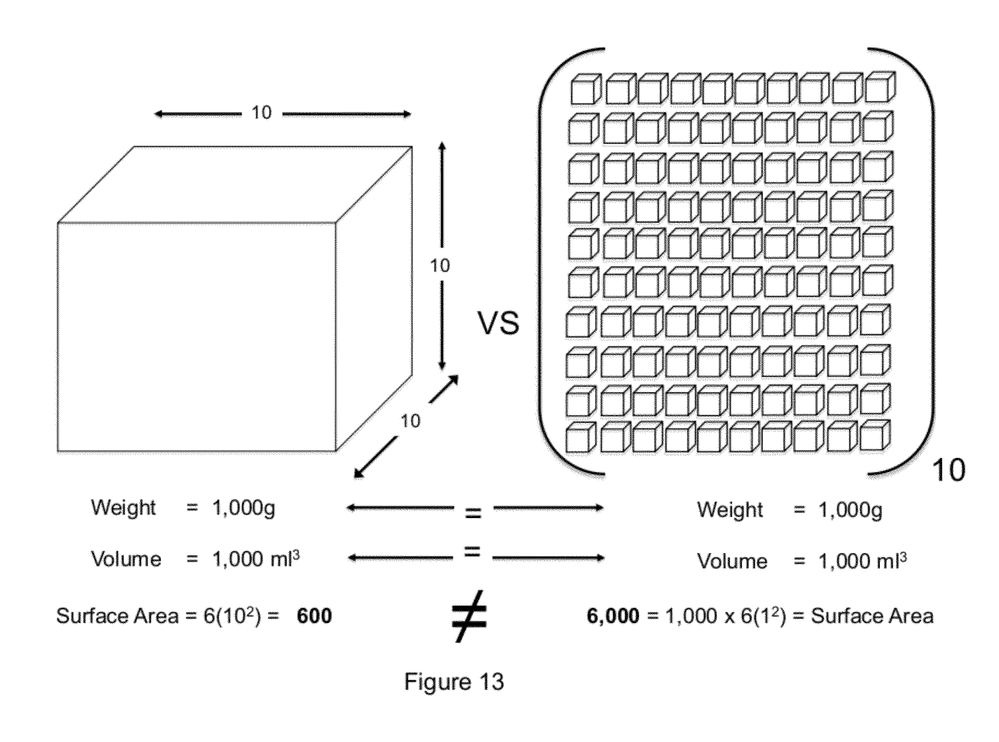

This clay having relative uniform distribution of particle size can be obtained, for example, by sifting hydrated sodium calcium aluminosilicate with a 325-mesh screen to separate and eliminate particles having sizes larger than about 45 microns. However, this type of separation method leads to the particle having an excess of static charge, which can be visualized by the sifted materials ability to electrostatically stick to a watch glass. FIG. 13 shows sifted particles (1320) having an electrostatic charge sticking to the watch glass (1310), whereas the Air classified particles (1330) have a much lower electrostatic charge and do not stick to the watch glass.

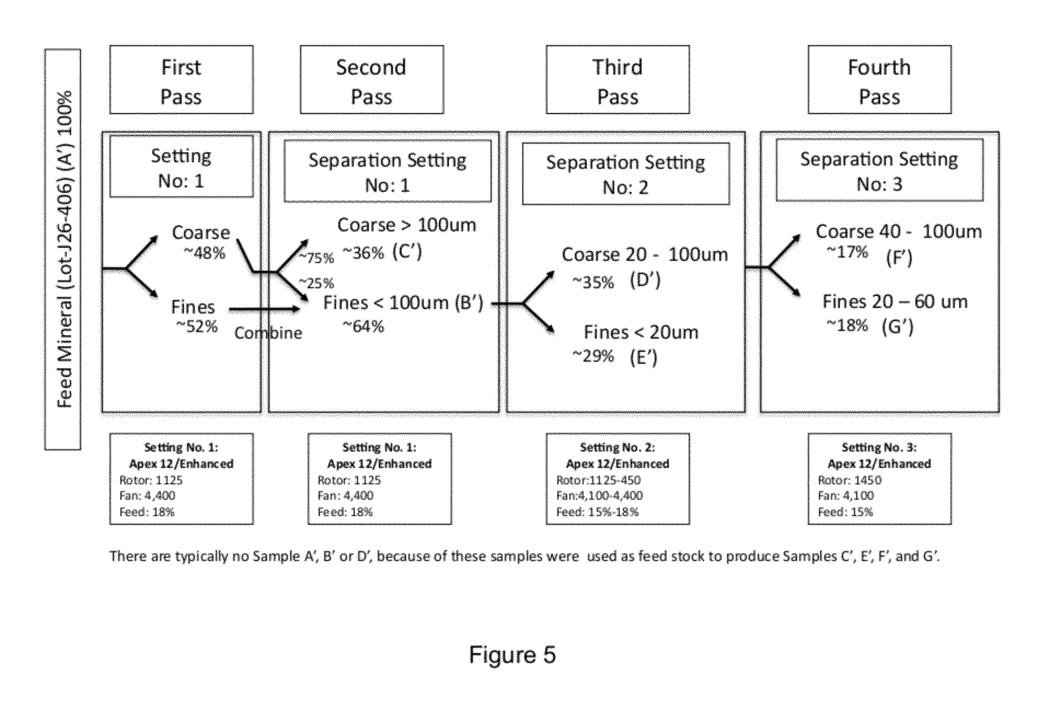

The feed stock clay was fed into the Air Classifier having the following settings No.:(1): Apex 12/Enhanced; Rotor: 1125; Fan: 4,400; Feed: 18%. The feed-stock was designated A′ and exited the Air Classifier as about a ˜48% “Coarse” Fraction having an average particle size that was greater than about 100 um, and a ˜52% “Fines” fraction having an average particle size that was less than about 100 um. The first “Coarse” fraction was run through the Air Classifier for a second pass with the same settings to yield another round of “Coarse” and “Fines” fractions. The first and second pass Coarse fractions were combined to form fraction C′ which represented about 36% of the original Feed Stock. The first and second pass Fines fractions were combined to form Fraction B′, which represented about ˜64% of the original feed stock. The Settings on the Air Classifier were set as follows when the fines fraction was sent through for a third pass—Setting No.:(2): Apex 12/Enhanced; Rotor: 1125-1450; Fan: 4,100-4,400; Feed: 15-18%. Fraction B′ exited the Air Classifier as about a ˜35% “Coarse” Fraction (D′) having an average particle size in the range of about 20 um to about 100 um, and a ˜29% “Fines” Fraction (E′) having an average particle size that was less than about 20 um. “Coarse” fraction (D) was run through the Air Classifier for a fourth pass with Setting No.:(3): Apex 12/Enhanced; Rotor: 1450; Fan: 4,400; Feed: 15% to yield another round of “Coarse” (F′) and “Fines” (G′) fractions. The Coarse fraction (F′) represented about 17% of the original Feed Stock, and “Fines” Fraction G′ represented about ˜18% of the original feed stock. The system is represented in FIG. 5.

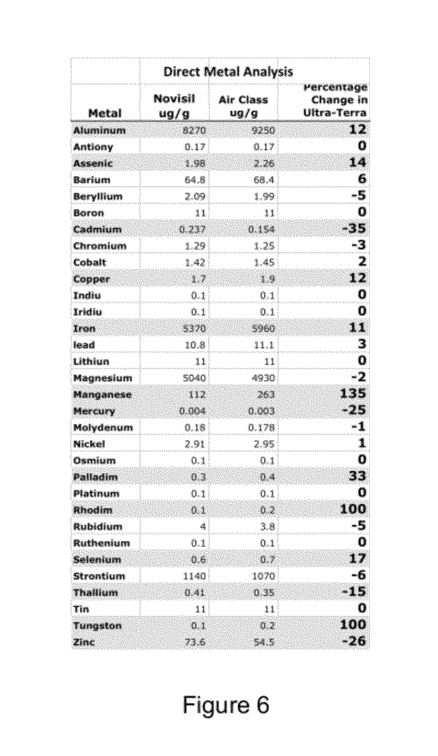

Some of the different fractions were subjected to metal analysis and compared to the feed stock using an independent analysis laboratory. The results are shown in FIG. 6. Some metals were found to be present in higher concentrations in the Air Classified fractions (e.g. Aluminum, Assenic Copper, Iron, Manganese, Palladium, Rhodim, Selenium, and Tungsten). In contrast, other metals were found to be higher in the feed material (e.g. Cadmium, Mercury and Zinc). As shown in FIG. 6, Cadmium was found be about 35% lower in the air-classified material. Lead was found to be about 25% lower, and Zinc about 26% lower.

One having ordinary skill in the art understands the structure function relationship between chemical compositions. More specifically, if the chemical structure is different, the chemical functions may also be different. Although not wanting to be bound by theory, the inventors submit that Air classification can produce a product that is chemically different than products alone.

### Example 2

### Description of Manufacturing Process and Process Controls

The montmorillonite clay that is discussed in this application has many names in the literature, for example, montmorillonite clay, bentonite clay, Hydrated Calcium AluminoSilicate (“HCAS”), and Hydrated Calcium Silicate (“HCS”). Clay from the Aberdeen, Miss. mine is processed to produce a product used for the examples. More specifically, the clay mined from the Aberdeen mine is processed to remove non-clay rocks and other debris and then transported by rail car to Jackson, Miss. The clay is then fed through a primary crusher and then processed through a secondary crusher to reduce the material size to ½ inch pieces or less. The material is then processed through a rotary dryer, which dries the HCAS material to 10%-14% moisture. The material is processed through a de-lumping screen and transferred to a feed bin. The feed bin transports the HCAS to a roller mill where the material is ground to the final size for the HCAS starting material. The HCAS is packaged in multi-walled paper bags.

During processing the clay starting material is crushed, sized, milled and dried. However, there are no solvents, reagents or catalysts used in the physical processing of the HCAS.

A portion of this starting material of HCAS is removed using the isolation described in Example 1. The air classified samples provide essentially pure clay with a particle size range of 20 μm-60 μm (>95%). There are no solvents, reagents or catalysts used in the physical processing of the HCAS at any point during this process of producing the raw materials for Distinct sized particle range products of montmorillonite.

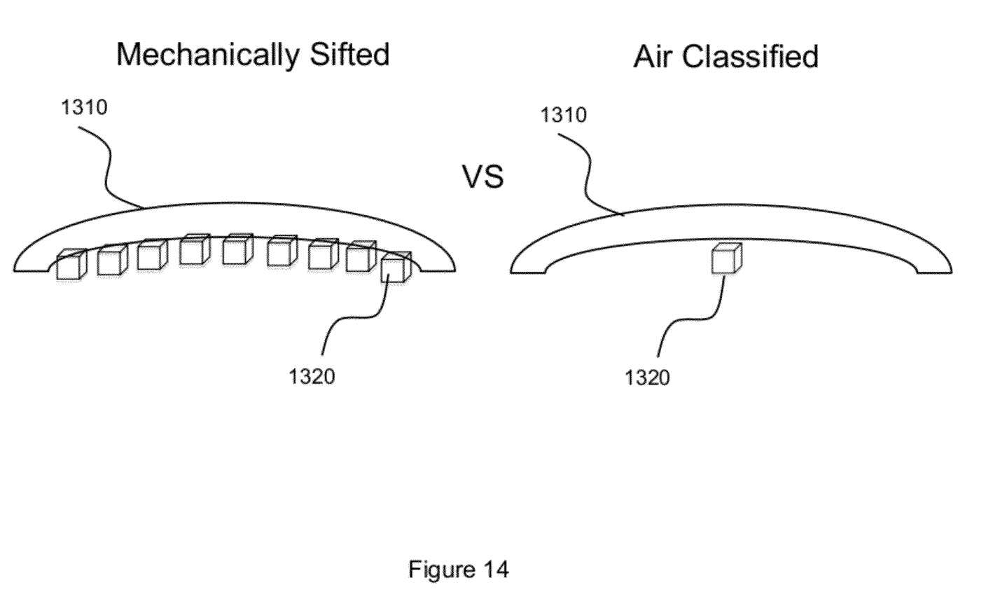

The flow diagram for the manufacturing process from mine to Hydrated Calcium Aluminosilicate and then to Distinct sized particle range products of montmorillonite are shown in FIG. 14. This flow diagram includes all steps in the process. However, the mining of the clay in Aberdeen, Miss. and the physical separation processing of the raw clay material at Jackson, Miss. do not need to be part of a system process.

### Environmental Background

Additionally, this isolation step results in a reduction of heavy metals as shown in FIG. 6 and dioxins as shown in Table 1. The chart below demonstrates that metal levels are altered in the isolated product.

TABLE 1
Shows the levels of Dioxins and Furans comparing Distinct sized particle range products
of montmorillonite with Novisil (“NS”). Additionally, the values for both NS and
Distinct sized particle range products of montmorillonite are well below the World
Health Organizations Tolerable Daily Intake (“WHO TDI”) limitations.

| | | Distinct sized | | TEQ (pg) per 3 g | |
|---|---|---|---|---|---|
| | | particle range | | Distinct sized | |
| | | products of | | particle range | |
| Dioxins/ | NS TEQ | montmorillonite | TEQ (pg) | products of | WHO TDI |
| Furans | (pg/g) | TEQ (pg/g) | per 3 g NS | montmorillonite | pg/kg BW/day |
| OCDD + | 0.0513 | 0.00307 | 0.154 | 0.009217 | 2.3 |
| HpCDD | | | | | |
| Other | ND | ND | — | — | |
| Dioxins | | | | | |

TEQ = Toxicity Equivalence for Dioxins/Furans
WHO TDI (Tolerable Daily Intake) is 2.3 pg/kg body weight/day for dioxins and furans.
BW = Body weight
ND = Not detected

### Example 3

### Alflatoxin Binding Data

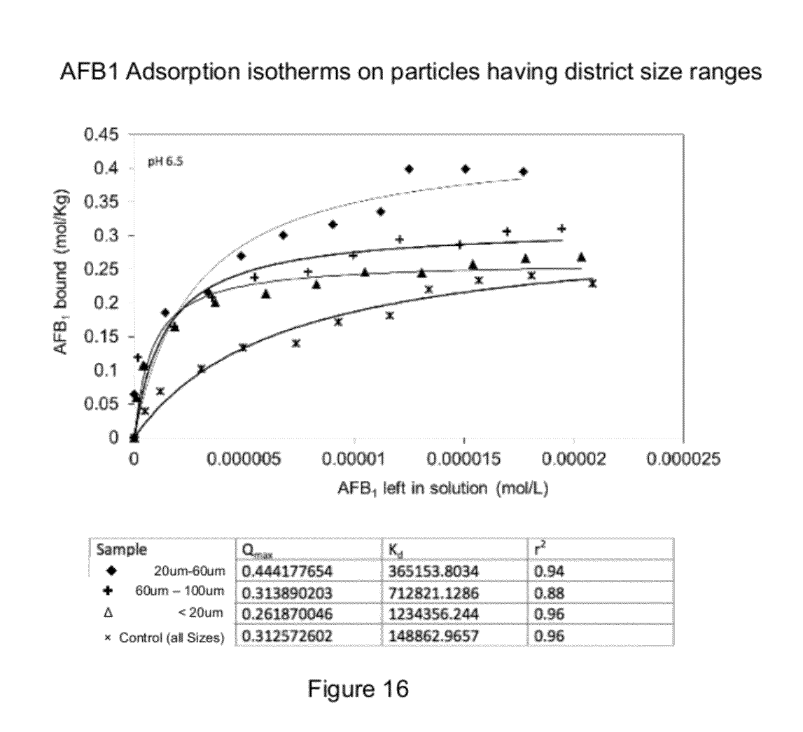

The innovative process selectively isolates components of a calcium montmorillonite from raw material purchased from the BASF mine (Jackson Miss.) into a different sized range product. More specifically, (a) less than 20 um; (b) 20 um to 60 um, (c) greater than 60 um; and (d) the starting feed stock with all sizes. Although not wanting to be bound by theory, a different chemically reactive product can be isolated from the calcium montmorillonite raw material that has a greater ability to bind aflatoxin AFB₁. More specifically, montmorillonite having a up to a 95% particle size range from 20 um-60 um has an aflatoxin AFB₁ binding Qₘₐₓ that is about (˜0.444). The aflatoxin AFB₁ binding Qₘₐₓ of the starting material is significantly lower about (˜0.313). The direct comparison of all sized products is shown FIG. 16. Particle ranges 60-100 um has a AFB₁ binding Qₘₐₓ ˜(0.31), and particle ranges less than 20 um has a AFB₁ binding Qₘₐₓ ˜(0.26).

The laboratory analysis shown in FIG. 16 was completed using the method outlined in Grant 1998, which is the reference cited in Appendix II of the Feed Industry Memorandum No. 5-23 and should be know by one having ordinary skill in the art.

The results from the isotherm analysis indicate that distinct particle sizes can be used at a lower rate of inclusion of aflatoxin binders in the feed and maintain efficacy in aflatoxin binding capacity when compared much greater amounts of the starting material. This limits weight and cost of producing and transporting these types of aflatoxin binders. More specifically, the distinct particle sized products have a more consistent particle size composition (˜20-60 um) when compared to starting material (˜5-300 um), which affords the (˜20-60 um) product with an increased surface area advantage that most efficiently bind aflatoxins and other mycotoxins on a weight per volume basis, as illustrated in FIG. 13. As a result, industrial consumers who purchase distinct sized particles will need to use less material for similar aflatoxin binding when compared to other products because the ability of the distinct sized raged particles to bind Aflatoxin is greater on a weight per volume basis.

### Example 4

### Resolution of Diarrhea

A 44 year old male having symptoms of diarrhea for at least 24 hours was treated every 6 hours with a dose of 0.5 grams of Product E′ over the time frame of 48 hours. Product E′ has an average particle size less than 20 um and fewer heavy metals (e.g. Cadmium, Mercury) when compared to the Stock Feed. Patient reported feeling better with partial diarrhea resolution within 6 hours and diarrhea was resolved in full by 18 hours.

A 46 year old male having a >10 year history of IBS and chronically having soft stool and/or diarrhea. Was treated every 12 hours with a dose of ˜0.75 grams of Product E′. Dramatic improvement in soft stool consistency within 24 hours, and practically eliminated bouts of diarrhea.

From this example it was possible to produce a general method of treating diarrhea. More specifically, a product isolated from example 1 can be used as a medicament for the resolution of soft stool or diarrhea a user. The product comprising a low cadmium containing calcium aluminum silicate product produced by the method of Example 1.

A method of resolving soft stool or diarrhea, comprises the following steps: (a) administering 0.1-1.0 g of an product produced by the method of claim 1 to a user in need of diarrhea resolution, wherein the product has average particle size under 100 um; (b) waiting a period of time, wherein the period of time is in the range of 0.1 hour to 6 hours; (c) repeating step (a) until the soft stool or diarrhea is resolved.

### Example 5

### Resolution of Diarrhea in Animals

A dog having a left sided anal sac mass, histiocytic sarcoma, hypercalcemia had Diarrhea for 4 days duration prior to presentation. Dog was admitted for hypercalcemia, vomiting, diarrhea and treated with 500 mg by mouth every 6 hours of Product G′ from FIG. 5. Diarrhea improved within 24 hours and resolved by 48 hours.

A pilot study having any canine patient with diarrhea persisting greater than 48 hours despite standard of care treatment with metronidazole was dosed with Product G′ (500 mg) by mouth every 8 hours. A total of 26 animals were treated for diarrhea—19 were chemotherapy induced diarrhea; 6 were non-chemotherapy related diarrhea. All but 3 cases were resolved.

What is claimed is:

1. A low cadmium containing calcium aluminum silicate product produced by the method of Air Classification, comprising the steps: (a) using an air classification system for separating a cadmium containing calcium aluminum silicate feed stock into at least a first fraction and a second fraction, wherein the first fraction contains material having an average particle size over 100 um, and the second fraction contains material having an average particle size under 100 um.

2. The product produced by the method of claim 1, further comprising the steps of:
(b) setting a rotor speed at about 1125; and (c) setting a fan speed at about 4,400.

3. The product produced by the method of claim 2, further comprising the step of: (d) setting a feed rate at about 18%.

4. The product produced by the method of claim 3, further comprising the step of: (e) making a second air classification separation of the second fraction containing material having an average particle size under 100 um and forming a third fraction and a fourth fraction, wherein the third fraction comprises particles having an average particle sized greater than 20 um, and the fourth fraction having an average particle size less than 20 um.

5. A product used as a medicament for the resolution of soft stool or diarrhea a user, the product comprising a low cadmium containing calcium aluminum silicate product produced by the method of claim 1.

6. A method of resolving soft stool or diarrhea, comprising the steps: (a) administering 0.1-1.0 g of an product produced by the method of claim 1, wherein the product has average particle size under 100 um; (b) waiting a period of time, wherein the period of time is in the range of 0.1 hour to 6 hours; (c) repeating step (a) until the soft stool or diarrhea is resolved.
